(19) United States
(12) Patent Application Publication
(10) Pub. No.: US 2012/0225493 A1
(43) Pub. Date: Sep. 6, 2012

(54) Electronic-Chemometric Controlled System and Process for the Analysis of Analytes

(75) Inventors: Brian D. Piorek, Santa Barbara, CA (US); Carl D. Meinhart, Santa Barbara, CA (US); Seung Joon Lee, Santa Barbara, CA (US); Casey Hare, Santa Barbara, CA (US); Norman Douglas Bradley, Santa Barbara, CA (US)

(73) Assignee: SpectraFluidics, Inc., Goleta, CA (US)

(21) Appl. No.: 13/390,664

(22) PCT Filed: Aug. 17, 2010
(86) PCT No.: PCT/US10/45761

§ 371 Date: May 10, 2012

Related U.S. Application Data
(60) Provisional application No. 61/234,926, filed on Aug. 18, 2009.

(51) Int. Cl. G01J 3/28; G01J 3/44; G01N 33/53; H01J 49/26

(52) U.S. Cl. 436/501; 250/339.01; 250/281; 250/372; 250/282; 356/301; 356/402; 422/69

(57) ABSTRACT

A series of electronic-chemometric control processes to enhance the selectivity, concentration, analysis, and detec tion of chemical species (analytes) in the gas phase, such as when using SERS-based techniques. Controls consist variously of: 1) feedback of electronic signals corresponding to changes of static and variable parameters in targeted chemical species that vary according to a reduction, increase, maximization, linearization, or improved confidence in one or more chemometric output parameters; 2) methods for spatially locating the source of an analyte species; and, 3) variable duty cycling to save power and materials according to altered physical and environmental conditions within a monitored zone.

### CROSS-REFERENCE

This application claims the benefit of U.S. Provisional Application No. 61/234,926, filed Aug. 18, 2009, which application is incorporated herein by reference.

### FIELD OF INVENTION

The invention relates to capturing airborne chemical species in the gas phase. More particularly, the invention relates to detection and/or analysis of low concentration chemical species using a fluid medium that transitions between vapor and liquid phases.

### BACKGROUND

Low concentrations of chemical species (analytes) targeted for detection and analysis pose unique technical challenges. Because low-concentration detection and analysis of some chemical compounds necessitate large and heavy lab apparatus, field deployment is often rendered difficult or impossible. In addition, the targeted analytes may be contaminated and/or mixed with false-positive compounds that confound accurate detection and analysis.

By definition, low concentrations generally represent a high ratio of inert or untargeted compounds to the targeted compound(s), often necessitating a process of filtering or other concentration processes, and with or without isolation or removal of contaminants. Thus, preparation is required to isolate and concentrate the analytes prior to the detection/analytic process, also inhibiting field portability.

There is a need for apparatus and processes that are both field portable and accurate, yielding minimal false-positive and false-negative detection events, and offering accurate and repeatable detection/analysis of the targeted analyte(s). Applications include hand-held chemical detectors for low-concentration analytes such as drugs, explosives, chemical and/or biological agents and weapons used in terrorist activities, and biological metabolites.

### SUMMARY OF INVENTION

The invention provides systems and processes suitable for analyzing and/or detecting airborne or gas-phase analytes. Various aspects of the invention described herein may be applied to any of the particular applications set forth below or for any other types of microfluidic or nanofluidic systems. The invention may be applied as a stand alone system or method, or as part of an integrated solution (such as in combination with a device or system described in International Patent Application No. PCT/US2010/34127, filed May 7, 2010, or International Application No. PCT/US2008/005345, filed Apr. 25, 2008, which are incorporated herein in their entireties), such as a portable analyte detection system. It shall be understood that different aspects of the invention can be appreciated individually, collectively, or in combination with each other.

In some embodiments, systems or devices described herein include hand-held chemical detectors for low-concentration analytes, such as those derived from drugs, explosives, and biological systems, having enhanced signal stability, accuracy, repeatability, and the ability to spatially locate analyte sources.

An aspect of the invention provides microfluidic devices and systems for various applications. Provided in certain embodiments herein is a system suitable for the detection and/or analysis of gas-phase analytes. In certain embodiments, an analyte detection system provided herein comprises (1) one or more microfluidic device; and (2) at least one module or control system configured to control or adjust one or more process or operation of the microfluidic device. In certain embodiments, the microfluidic device comprises (1) one or more microfluidic chambers comprising therein a condensed liquid medium; and (2) an analytical instrument suitable for providing an identifiable analytical output (e.g., a signal or spectrum of an analyte). Specifically, the microfluidic chamber comprises at least one opening (or open surface) that provides an interface or contact between the gas-phase (e.g., a gas-phase comprising, or potentially comprising, an analyte of interest) and the liquid medium. Preferably, the interface or contact between the liquid medium and gas-phase allows for evaporation of the condensed liquid medium, and subsequent re-condensation thereof. In some embodiments, microfluidic devices described herein operate according to at least one variable operating parameter and, optionally, at least one static (non-variable) operating parameter.

Specifically, an analyte detection system provided herein further comprises (3) at least one module configured to adjust one or more variable operating parameters of the microfluidic device. In specific embodiments, at least one module is configured to adjust the one or more variable operating parameters based on the results of the chemometric processing of at least one output of the microfluidic device.

In certain embodiments, outputs of the microfluidic device may include, by way of non-limiting example, an analytical output, a measured parameter output, or the like, or a combination thereof. In specific embodiments, an analytical output is an output based on the interrogation of an analyte with an analytical instrument. In specific embodiments, the analytical output is a spectrum (or portions thereof), spectra (or portions thereof), or the like. In more specific embodiments, the analytical output is a SERS spectrum (or portions thereof), or SERS spectra (or portions thereof). In some embodiments, a measured parameter output may be a measured operating parameter of the microfluidic device. In preferred embodiments operating parameters are adjusted based on data derived from chemometric analysis of an analytical output such as a SERS spectra. In specific embodiments, a measured parameter output may include, by way of non-limiting example, a duty cycle rate of one or more microfluidic chambers of the microfluidic device, a flow rate of one or more microfluidic channels of the microfluidic device, the air flow rate within the microfluidic device, or any other operating parameter of the microfluidic device. In certain instances, the measured parameter may be different from the input parameter because, e.g., of interactions between various air-borne chemicals (e.g., analytes) and the components of the microfluidic device.

In some embodiments, the module configured to chemometrically process at least one output compares data output from the microfluidic device (e.g., an identifiable signal or spectrum of an analyte and/or measured operating parameters of the device) to a set of data or database (e.g., a database comprising a library of known analyte signals and/or spectra, and/or operating parameters). In certain embodiments, comparison of analyte signals or spectra obtained from analytical instruments of a system described herein comprises comparison of signal peaks, signal troughs, intensity of signal peaks, lack of signal peaks, breadth of signal peaks, or the like of at least one spectrum measured by a system described herein to a database library (e.g., either a database comprising complete spectra and/or various signal peaks, signal troughs, intensity of signal peaks, lack of signal peaks, breadth of signal peaks, or the like of spectra of known analytes). In some embodiments, the dataset or database further or alternatively comprises operating parameters of the microfluidic device. In some embodiments, the output parameter(s) chemometrically processed are subject to a more complex analysis in comparing such output(s) of the microfluidic device to a database or dataset (e.g., a library of known analytes that includes various spectral data thereof and/or operating parameters corresponding thereto). For example, in some embodiments, chemometric processing of the one or more outputs is compared to a library using a Routh array. A preferred output of the chemometric analysis module is the identification of specific analytes present in the microfluidic system, based on analyzing the output, such as a SERS spectra. In this case the operating parameters may be adjusted to control, i.e., increase, decrease, maintain or any combination the amount of specific analyte(s) determined by chemometric analysis of the analytical output (SERS spectra for example).

In some embodiments, the microfluidic device is integrated with one or more modules of the analyte detection system. For example, in some embodiments, the microfluidic device of the analyte detection system comprises a processor comprising at least one module configured to control or adjust one or more processes or operations of the microfluidic device and/or other modules present. In other embodiments, an analyte detection system described herein may optionally have a processor comprising one or more modules of the system external to the microfluidic device.

In certain instances (e.g., following the open surface of the chamber being brought into proximity or contact with a gas-phase analyte), a microfluidic chamber of a microfluidic device described herein comprises therein a fluid (e.g., a condensed liquid medium) and an analyte. In certain embodiments, the microfluidic device comprising a condensed liquid within one or more partially exposed microfluidic chambers therein can be subjected to a series of cyclical flooding and evaporation cycles. One or more chambers may be filled with a desired fluid at selected intervals. A number of evaporation cycles may be performed, thus volumetrically concentrating the targeted analytes within the liquid-phase fluid.

As provided herein, certain analyte detection systems of the present invention comprise microfluidic device(s) and analytical system(s), such devices or systems provide an analytical output, e.g., a SERS output. Generally, devices that are suitable for providing a SERS output comprise a SERS active surface from which SERS interrogation can occur. Accordingly, in some embodiments, a microfluidic chamber of a microfluidic device described herein comprises therein a fluid (e.g., a condensed liquid medium), an analyte, and nanoparticles comprising a SERS-active surface. In specific embodiments, a microfluidic device described herein comprises one or more microfluidic chambers with colloidal nanoparticles therein. In some embodiments, the colloidal nanoparticles are aggregated colloidal nanoparticles. In specific embodiments, the aggregated nanoparticles are aggregated with an analyte and/or a second nanoparticle. In some embodiments, the one or more microfluidic chambers comprise one or more microfluidic channels, one or more microfluidic cells with at least one opening or surface exposed to a gas phase environment, or a combination thereof. In specific embodiments, the one or more microfluidic chambers comprise one or more microfluidic cells.

In some embodiments, the analytical instrument of a system described herein is any analytical instrument capable of providing an analytical output when configured with such a system. In specific embodiments, an analytical instrument provided for herein is, by way of non-limiting example, a surface enhanced vibrational spectrometer, surface plasmon resonance spectrometer, a system based on electrochemical analysis techniques which may include molecular recognition elements, a system based on fluorescent chemical marker techniques, a system based on fluorescence quenching, a system based on redox-labeled nucleic acid binding techniques (including, but not limited to, the molecules DNA, RNA and PNA), a system based on X-Ray absorption techniques, IR spectrometer, visible analytical techniques, UV spectrometer, a system based on other electromagnetic radiation absorption techniques, mass spectrometer, a system based on liquid chromatography techniques, a system based on flame ionization analysis techniques, a system based on DNA melting point techniques, or a system based on titration analysis techniques. In some embodiments, the analytical instrument is a Raman spectrometer (e.g., a Raman spectrometer suitable for surface-enhanced Raman spectroscopy (SERS)).

As discussed above, certain analyte detection systems of the present invention comprise microfluidic devices and analytical instruments, such devices or systems provide an analytical output, e.g., a SERS output. However, the detection of extremely low concentrations of chemical species (analytes), such as via surface-enhanced Raman spectroscopy (SERS), poses a number of practical challenges related to sampling. In some cases, active regions may yield spectral data over discrete intervals of time that limit the practical sampling rate of SERS. In addition, the delayed aggregation rate of colloidal nanoparticles in the presence of analyte(s) may not accurately reflect real-time changes in analyte concentration within specimens and/or the test environment, thus introducing lag and dwell errors. In some instances, therefore, without some form of feedback to compare changes in analyte concentration of colloidal nanoparticles over intervals of time, SERS enhancement and the sensitivity of subsequent detection may be of limited fidelity. In certain instances, this limitation is due in whole or part to the requirement for the appropriate development of ‘hot’ SERS colloidal aggregates, e.g., dimers, to occur within the SERS interrogation region and during SERS interrogation for detection to occur. In some instances, the aggregation rate of the colloidal nanoparticles varies with analyte species.

In some embodiments, an analyte detection system, preferably comprising an analytical instrument and a chemometric processing module described herein comprises at least one module that controls or adjusts one or more variable operating parameter(s) (e.g., microchannel flow rate or microcell duty cycle rate) of a microfluidic device in order to ensure the analyte-induced ‘hot’ SERS colloids do not aggregate before exposure to an interrogation laser (overaggregation) of a Raman spectrometer, or after exposure to the interrogation laser (underaggregation) (e.g., when passing through a microchannel for detection). Other events, such as fluctuations in detection events, chemical noise and/or changing environmental factors may also limit the practical application of this detection technique without one or more modules or a system that controls or adjusts one or more operating parameter(s) of a microfluidic chamber or device or chamber thereof.

In certain embodiments, the analyte detection system uses a feedback control application to further facilitate analyte detection. In some embodiments, a feedback control application comprises (1) at least one module configured chemometrically to process one or more outputs of the microfluidic device (e.g., an identifiable signal or spectrum of an analyte and/or measured operating parameters of the device), such as by comparing the output to a set of data or database (e.g., a database comprising a library of known analyte signals and/or spectra, and/or operating parameters); and (2) at least one module configured to adjust the variable operating parameters of a microfluidic device described herein, e.g., based on the comparison of the microfluidic output (e.g., spectra or portions thereof) to such a database or dataset. In certain embodiments, the feedback control application allows the process of comparing output data to stored data and a subsequent adjustment of the variable operating parameters of the microfluidic device to be repeated one or more times, e.g., until a desired or optimal result is obtained. In some embodiments, adjustment of the variable operating parameters based on the comparison of the microfluidic output to such a database or dataset involves comparing a spectrum, or portion thereof, of a detected analyte to a database of spectra, or portions thereof, finding a similar spectrum, or portion thereof, in the database, and adjusting one or more variable operating parameters of the microfluidic device to correspond to the operating parameter(s) corresponding to (e.g., used to obtain) the stored spectrum, or portion(s) thereof. In certain embodiments, the database comprises the spectrum, or portion thereof, of a known analyte and the corresponding operating parameters utilized to obtain the stored spectrum or other corresponding operating parameters previously determined to characteristically adjust, improve, and/or optimize the analytical detection thereof.

In certain embodiments, provided herein is a system comprising a processor comprising one or more modules, incorporating feedback, for detecting real-time changes, based on chemometric data, in aggregation rates of colloidal nanoparticles, thus conferring: 1) enhanced signal stability; 2) optimized signal-to-noise ratio; 3) the ability to measure relative and/or absolute changes in concentration of analytes; and/or 4) the ability to spatially hunt for the physical location(s) of high-magnitude SERS signals resulting from the interaction of analytes with colloidal nanoparticles.

In one embodiment, feedback comprises signal or spectral processing (e.g., analog or digital processing) which analyzes data from SERS/Raman-based spectra (i.e., chemometrically processed data (FIG. 1), and/or non-processed data). The feedback modifies a number of variable parameters which, in turn, adjust one or more variable operating parameters within the instrument, such as to:

a. Reduce one or more outputs (e.g., chemometric output);

b. Increase one or more outputs (e.g., chemometric output);

c. Maximize one or more outputs (e.g., chemometric output);

d. Linearize one or more outputs (e.g., chemometric output); and/or

e. Improve confidence in one or more outputs (e.g., chemometric output).

In some embodiments, a system described herein has certain static parameters that are not adjusted or controlled by a module or chemometric processor described herein. In certain embodiments, such static parameters may include, by way of non-limiting examples:

a. Width of microfluidic channel(s) and/or cell(s); and/or

b. Depth of microfludic channel(s) and/or cell(s).

In some embodiments, a system described herein has certain variable operating parameters that may be adjusted or controlled by a module or chemometric processor described herein. In certain embodiments (e.g., in SERS detection), variable operating parameters may include, by way of non-limiting example:
a. Rate of condensation-evaporation cycling within microfluidic channel(s) and/or cell(s);
b. Flowrate of colloid within microfluidic channels;
c. Power level of interrogating laser;
d. Wavelength of interrogating laser;
e. Flow rate of sampled air (fluid in a gaseous phase);
f. Integration time of the Raman spectrometer;
g. Relative humidity of the sample gas;
h. Nanoparticle size within colloid;
i. Nanoparticle size deposited on the microchannel surface or substrate;
j. Nanoparticle density deposited on the microchannel surface or substrate;
k. Nanoparticle concentration in the working fluid;
l. Chemical composition of working fluid, which may contain nanoparticles;
m. Operating temperature;
n. Background fluorescence spectra; and/or
o. Photodecomposition fluorescence spectra.

In alternative embodiments, systems may be configured such that any one or more of such parameters may, instead, be a static parameter.

Moreover, in some embodiments, one or more laser wavelengths and power settings may be employed, wherein SERS spectra from the targeted analyte(s) are analyzed along with background and photodecomposition fluorescence spectra.

In one embodiment, spectra and environmental conditions within the monitored zone are correlated with the spatial location of the detection apparatus (FIG. 3). This process is useful for spatially locating (hunting) the physical location of an analyte's source.

In one embodiment (FIG. 4), a variable duty cycle enables the detection device at periodic intervals of time to more efficiently monitor zones such as cargo containers. Duty cycling may be adjusted according to environmental conditions, entry to the zone and/or changes in monitored contents (FIG. 5), whereupon the detection device—responding to the presence of targeted analyte(s)—telemeters data to a receiving station. Intelligent duty cycling saves power and materials.

In one embodiment (FIG. 6), a library of parameters and/or spectral data reside in an electronic medium, such as a programmable read-only memory (PROM) or random access (RAM) device, along with the substrate-based SERS-active regions, in an integrated, interchangeable device. This provides a set of resident, static data parameters, which may be compared with variable operating parameters found within the monitored environment to adjust the variable operating parameters of the microfluidic device.

In one embodiment, the parameters are adjusted to effectively ‘zero out’ the signal. This is particularly useful after a detection has been made, but also while a signal is still being observed by the system. Proper feedback control of one or more parameters can be adjusted, such that the system is effectively ‘zeroed out’ to provide an effective baseline for accurate, calibrated measurements.

In a preferable embodiment of the invention, a microfluidic system may comprise a device formed with a plurality of microfluidic cells and/or microcells. The cells may be preferably formed with a diameter ranging from approximately 10 nanometers to 1000 micrometers, or from about 10 nanometers to about 200 nanometers. In specific embodiments, the system comprises a microfluidic device and/or one or more microfluidic chambers as described in International Patent Application No. PCT/US2010/34127, filed May 7, 2010, or U.S. Patent Application No. 61/176,473, filed May 7, 2009, which are hereby incorporated herein by reference in its entirety. In another embodiment, a device or system provided for herein may be formed with a plurality of microfluidic channels. In specific embodiments, the system comprises a microfluidic device or one and/or more microfluidic chambers as described in International Application No. PCT/US2008/005345, filed Apr. 25, 2008, which is hereby incorporated herein by reference in its entirety.

A selected liquid such as water may be contained or confined over nanostructured surfaces within the microfluidic cells, which interact with a targeted analyte, either chemically or physically. The liquid may be selected for its relative affinity or repulsiveness to a particular analyte or class of analytes, thus substantially excluding contaminants and/or non-selected chemical species, thereby facilitating desired concentration and specificity for the analyte.

Microfluidic devices provided herein comprise an air/liquid interface providing selectivity for a targeted molecule. In some instances, selectivity occurs by allowing polar molecules to partition into the aqueous liquid and non-polar molecules to not partition into the liquid. In certain instances, this is a result of the relative values of Henry's constants between various analyte molecules. The condensed liquid medium provides concentration of the analyte molecules, which may be quantified by the absolute value of the associated Henry's constant. The level of concentration can be significant under equilibrium conditions, but it may take a significant amount of time to reach equilibrium. Thus, in some embodiments, a microfluidic device provided herein comprises a mechanism (e.g., one or more components or device) for active cycling of the liquid/vapor exchange (the rate of the cycling being referred to herein as the duty rate).

As analytes interact with formed nanostructures within a liquid-phase fluid, they can be detected and/or analyzed using a variety of technologies. For example, the analytes may undergo or be studied using methods such as surface enhanced vibrational spectroscopy, surface plasmon resonance spectroscopy, electrochemical analysis techniques which may include molecular recognition elements, fluorescent chemical marker techniques, fluorescence quenching, redox-labeled nucleic acid binding techniques (including, but not limited to, the molecules DNA, RNA and PNA), X-Ray absorption techniques, IR, visible, UV, and other electromagnetic radiation absorption and spectroscopic techniques, mass spectroscopy techniques, liquid chromatography techniques, flame ionization analysis techniques, DNA melting point techniques, or titration analysis techniques.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The foregoing features and other aspects of the invention are explained in the following description taken in conjunction with the accompanying figures. Further understanding of the features and advantages of the invention will be obtained by reference to the following detailed description that sets forth illustrative embodiments, in which the principles of the invention are utilized.

FIG. 1 illustrates a process of chemometrically processing an output of a microfluidic device and a feedback application of using this processed output to adjust a variable operating parameter of a microfluidic device in order to improve the analytical output of the device and, thereby, increase the probability of the device in properly identifying an analyte detected in the microfluidic device.

FIG. 2 illustrates a leveling-off of analyte SERS signal strength due to a transition from dimer to trimer aggregation of analyte within a nanoparticle-bearing colloid or upon a nanoparticle-deposited substrate. In certain embodiments, adjustment of one or more variable operating parameters of a microfluidic device as described herein allows for a decrease in the formation of trimers at the site of SERS interrogation, thereby providing for an increase in SERS signal strength with analyte gas concentrations that would otherwise produce trimers using non-adjusted operating parameters.

FIG. 3 illustrates how the source of a targeted analyte may be spatially located by taking a series of comparative measurements in its vicinity.

FIG. 4 illustrates deployment of the detection device in a space where long-term monitoring may render intelligent duty cycling controls desired in order to save power and materials.

FIG. 5 illustrates an active evolution of the device shown in FIG. 4. Entry to the monitored space, presence of a targeted analyte (117), and/or periodic duty cycling enables the detection device for sensing (118). If the targeted analyte(s) are detected, a response is triggered, such as activation of an alarm or telemetering of data (119).

FIG. 6 illustrates a SERS sensor module having an integrated PROM containing a data library and an array of active sites.

### DETAILED DESCRIPTION OF INVENTION

While preferable embodiments of the invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

Provided in certain embodiments herein is a microfluidic system for capturing and analyzing gas phase and/or airborne analytes in a liquid, comprising:
at least one liquid/gas interface site, comprising at least a partially open microchamber, including at least one of a microchannel or a microcell,
at least one analytical instrument
at least one chemometric data processing module configured to process outputs from the analytical instrument; and,
at least one system control module configured to adjust one or more variable parameter;
   wherein at least one operating parameter of the microfluidic system is adjusted based on an output from the chemometric processing module. In certain embodiments, the microfluidic system further comprises at least one of one or more nanostructured surfaces in the microchamber or provision for the system to introduce nanostructured particles into the microchamberIn specific embodiments, the system further comprises at least one processor configured to execute the chemometric data processing module and/or the system control module.

Furthermore, provided in some embodiments herein is a process of detecting or measuring the amount of a gas phase and/or airborne analyte molecule in an air sample utilizing a microfluidic system, the process comprising:
providing at least one partially open microchamber comprising a liquid and a liquid surface that is in contact with the air sample;
exposing the sample of air with the surface of the liquid, thereby allowing analyte molecules to diffuse into the liquid in one or more of the microchambers;
interrogating the microcell with an analytical instrument to acquire data pertaining to the analyte molecules;
analyzing the acquired analytical instrument data with a chemometric data processing module; and,
adjusting at least one operating parameter of the microfluidic system based on an output from the chemometric data processing module.

In some embodiments, either the chamber and/or the liquid includes nanostructured material upon diffusion into the liquid, the analyte aggregates with and/or is deposited on the nanostructured material.

The foregoing features and other aspects of the invention are explained in the following description taken in conjunction with the accompanying figures, wherein:

FIG. 1 illustrates one embodiment of the invention wherein real-time or discretized output data from SERS-based spectra taken of an unknown analyte from a monitored zone of a microfluidic device or chamber are compared with a set of stored data (e.g., a library of data comprising various spectral data and/or operating parameters, as described herein). Thus, in certain embodiments, a system described herein comprises a module configured to chemometrically compare real-time or discretized output data (e.g., from an analytical signal or spectrum (or spectra) or one or more measured operating parameter) to a set of data. In certain embodiments, such data includes, by way of non-limiting example, stored data (e.g., non-processed or discretized chemometric) from SERS-based spectra of a known analyte and/or the operating parameters used to obtain such data, or measured operating parameters of the device. Any suitable module may be utilized including ones processing analog or digital data.

In some embodiments, using feedback calculations, chemometric processing of output data (e.g., processing of a comparison or the difference between measured data) and stored data provides variable operating parameters which, in turn, are utilized in the microfluidic device thereby adjusting one or more internal states within the device. In certain embodiments, these calculations are utilized to adjust or modify one or more variable operating parameter(s) of the device. Thus, in some embodiments provided herein is a system comprising a module configured to adjust one or more variable operating parameter(s) of the microfluidic device based on the comparison of real-time or discretized chemometric data from a device output (e.g., analytical data and/or measured operating parameters) to a set of data (e.g., stored or measured data). Thus, the qualitative output of the system can be adjusted to correspond to the presence and chemical characteristics of one or more targeted analytes.

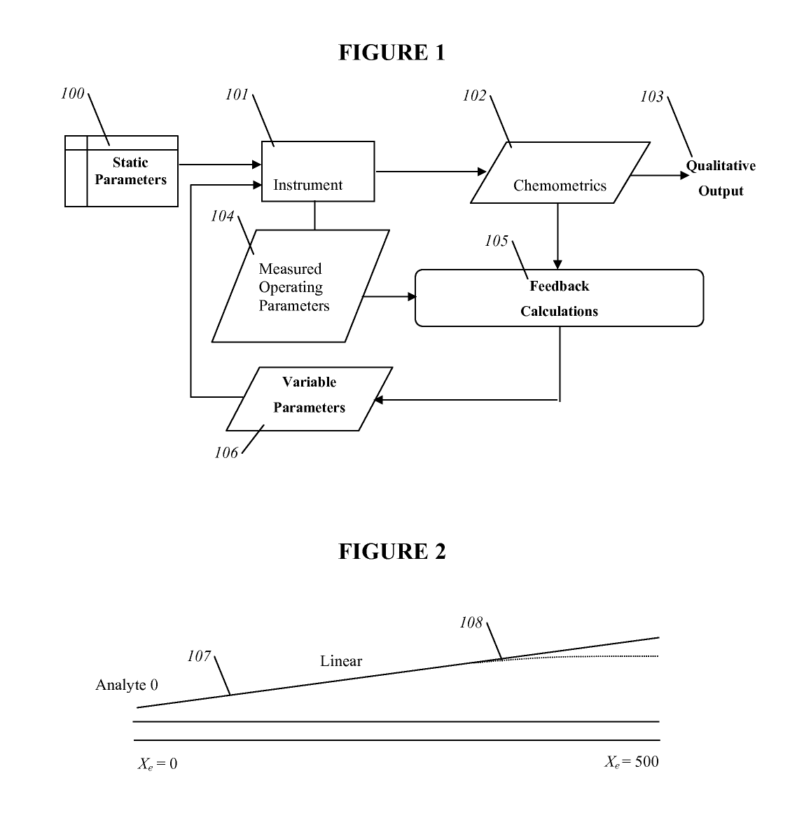

FIG. 2 illustrates how, in certain instances, linear data (107), corresponding to increased SERS spectra with an increasing presence of analyte, demonstrates a leveling-off (108) due to a transition from dimer to trimer aggregation of analyte within a nanoparticle-bearing colloid or upon a nanoparticle-deposited substrate. This is one embodiment wherein chemometric data provide parameters for feedback control.

In certain embodiments, a microfluidic device described herein has as a variable operating parameter, and a module configured to adjust the rate of condensation-evaporation cycling within at least one microfluidic chamber (e.g., at least one microfluidic channel and/or cell) of the microfluidic device. In certain embodiments, the rate of the condensation-evaporation cycling within the microfluidic chambers of microfluidic devices described herein affects the rate at which analyte is captured into the microfluidic chamber.

In certain embodiments, active cycling of the liquid/vapor exchange includes actively evaporating the liquid (i.e., the condensed form of the fluid used in a device described herein, such as, e.g., water) and/or actively condensing the vapor (i.e., the evaporated form of the fluid used in a device described herein, such as, e.g., water). Active cycling of the liquid/vapor exchange can be achieved utilizing any suitable component, device or process. In specific embodiments, active cycling is achieved, e.g., through any active pumping process, including, by way of non-limiting example, heating and/or cooling processes or cycling, reduced and/or elevated pressure processes or cycling, or the like. In certain embodiments, evaporating the fluid (e.g., a solvent of the analyte) and condensing the fluid (e.g., a solvent of the analyte) are performed concurrently, sequentially, alternately, or the like. In some instances, the time constraint to reach equilibrium conditions can be reduced substantially by active pumping of the liquid/vapor exchange at the free surface. This “active pumping” can be achieved in any suitable manner, including, e.g., temporally cycling the local temperature of the liquid region above and below the ambient dew point. Generally, and in preferred embodiments, analyte molecules (e.g., targeted or selected molecules) that are captured in the liquid do not evaporate at the same rate as the liquid evaporates. Thus, in certain embodiments, the analyte molecules (e.g., targeted or selected molecules) remain in one or more of the chambers (e.g., cells or channels) and are available for detection (e.g., in some instances, the molecules adsorb to a surface-enhanced Raman scattering (SERS) active surface such as one or an assembly of nanoparticles or nanowires/nanorods, or any other suitably nanostructured metal surfaces, or an assembly of nanoparticles onto metal or non-metal substrate surfaces) within a microfluidic device described herein).

In certain embodiments, a microfluidic device described herein has as a variable operating parameter, and a module configured to adjust, the flowrate of colloid within microfluidic channels. The flowrate of a colloid within a microfluidic channel may be achieved in any suitable manner. For example, the flowrate of the microfluidic channel may be adjusted, thereby adjusting the flowrate of the colloid within the microfludic channel. Alternatively, a device described herein may have a plurality of microfluidic channels operating at different flowrates. Thus, in certain embodiments, the analyte may be analyzed with an analytical instrument, as described herein, the analyte detected being in a microfluidic channel having a flowrate different from the microfluidic channel in which the analyte is originally analyzed.

In certain embodiments, a microfluidic device described herein has as a variable operating parameter, and a module configured to adjust, one or more parameter of an analytical instrument (e.g., Raman spectrometer). In various embodiments, the power level of an interrogating laser, the wavelength of interrogating laser, integration time of the analytical device (e.g., Raman spectrometer), or a combination thereof, may be adjusted.

In certain embodiments, a microfluidic device described herein has as a variable operating parameter, and a module configured to adjust, the flow rate of sampled air (fluid in a gaseous phase) present in the device. For example, in certain embodiments, a device may comprise a compartment surrounding one or more microfluidic chambers, the compartment being open to the air to be sampled by one or more inlets, the size of the inlet(s) being adjustable. In other embodiments, the device may comprise a fan or pump with an adjustable rpm that may be used to vary the flow rate of the sampled air.

In certain embodiments, a microfluidic device described herein has as a variable operating parameter, and a module configured to adjust, the relative humidity of the sample gas. This adjustment may be achieved in any suitable manner. For example, a system or microfluidic device described herein may optionally comprise a humidifier (e.g., a variable humidifer).

In certain embodiments, a microfluidic device described herein has as a variable operating parameter, and a module configured to adjust, the nanoparticle size in the colloid, nanoparticle size deposited on a microfluidic chamber surface or substrate, nanoparticle density on a microfluidic chamber surface or substrate, nanoparticle concentration in the fluid of the microfluidic chamber, a like nanoparticle variance, or a combination thereof. Variance of any nanoparticle variable utilized in a system described herein may be adjusted in any suitable manner. For example, in certain embodiments, a microfluidic device described herein may comprise a plurality of storage compartments comprising a variety of different nanoparticles any one of which may be inserted into a microfluidic chamber (e.g., microfluidic channel) to be interrogated, depending on the variable operating parameter input by the module configured to adjust the nanoparticle variable (e.g., size, concentration, or the like) within the colloid, chamber, liquid, or the like. In other embodiments, a microfluidic device described herein may have a plurality of microfluidic chambers comprising nanoparticles, wherein at least two of the microfluidic chambers comprise nanoparticles in different sizes, concentrations, densities, or the like. Thus, in certain embodiments, the analyte is first detected in a first microfluidic channel or chamber having a first nanoparticle characteristic (e.g., having a given set of nanoparticle size in the colloid, nanoparticle size deposited on a microfluidic chamber surface or substrate, nanoparticle density on a microfluidic chamber surface or substrate, nanoparticle concentration in the fluid of the microfluidic chamber, and like nanoparticle characteristics) and, following feedback, is detected in a secondary microfluidic channel or chamber having a second nanoparticle characteristic.

In certain embodiments, a microfluidic device described herein has as a variable operating parameter, and a module configured to adjust, the chemical composition of working fluid, which may contain nanoparticles. In some embodiments, adjustment of the chemical composition is achieved by utilizing a microfluidic device with various reservoirs of fluids which may input into one or more microfluidic chambers of the system depending on the desired adjustment. Moreover, in certain embodiments, a microfluidic device described herein may comprise at least two different microfluidic chambers, the first of which comprises a first working fluid and the second of which comprises a second working fluid. In specific embodiments, these first and second chambers may be in discrete sections (e.g., so as to avoid mixing of the vapors of the chambers).

In certain embodiments, a microfluidic device described herein has as a variable operating parameter, and a module configured to adjust, the operating temperature of the microfluidic device and/or microfluidic chamber. Adjustment of the operating temperature may be achieved in any suitable manner. In specific embodiments, operation temperature may be adjusted using heating elements, a laser, or the like.

In certain embodiments, a microfluidic device described herein has as a variable operating parameter, and a module configured to adjust, the background or blank spectra (e.g., a backgroud UV-Vis, fluorescence, or the like spectra), for the particular analytical instrument utilized. In specific embodiments, a new background or blank spectra, or a stored background or blank spectra may be utilized.

In certain embodiments, a microfluidic device described herein has as a variable operating parameter, and a module configured to adjust, the photodecomposition fluorescence spectra.

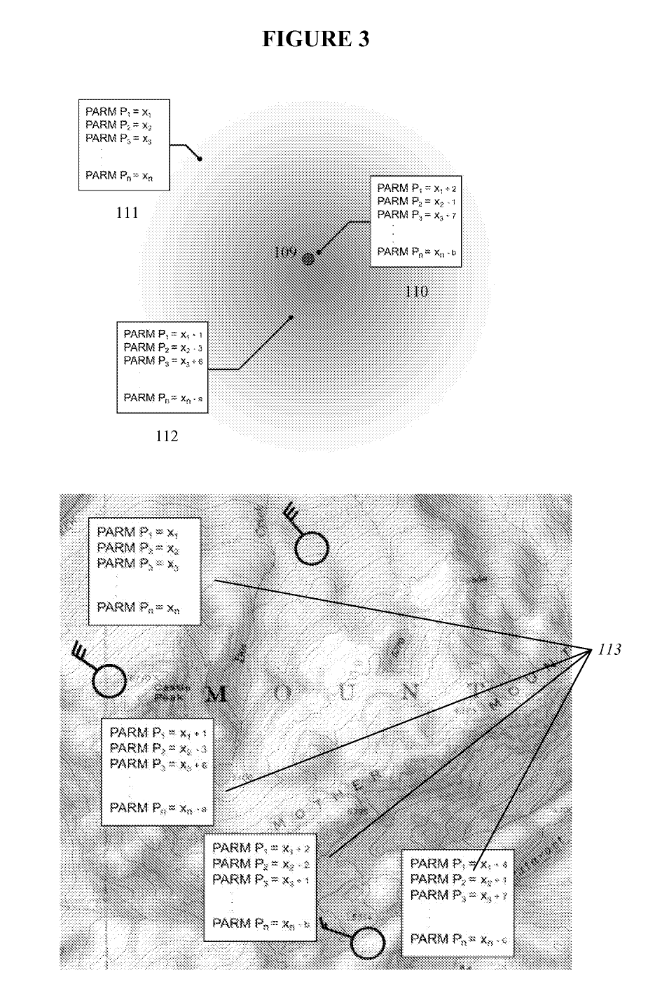

FIG. 3 illustrates how the source of a targeted analyte (109) may be spatially located by taking a series of comparative measurements in its vicinity. Since concentration and chemical characteristics of gas- or liquid-borne analyte are proportional to the analyte's rate of diffusion from the source through a surrounding medium, a relationship may be discerned between the locations of said measurements and the source itself (110-112). Measurements may be collected as real-time, streaming analog data, and as discretized (e.g., digital) data deriving from SERS, fluorescence, or photodecomposition spectra, and corresponding to the presence or saturation of analyte within the monitored zone; data may be rendered in raw form, as time- or spatial-domain variables, and stochastically. By incorporating additional environmental data (e.g., wind speed and direction in macro environments), parametric data collected by the detection device may be interpreted to render stochastic output corresponding to the nature, location and concentration of the targeted analyte source(s) (113).

In some embodiments, a microfluidic device described herein has standard or default operating parameters that provide for long periods of inactivity of one or more processes of the microfluidic device. In certain embodiments, the microfluidic device periodically analyzes the gas phase therein for a gas-borne analyte. In some embodiments, such a microfluidic device comprises: (1) a module configured to compare measured data (e.g., chemometrically processed or unprocessed data) received from such an analysis (e.g., a spectrum or portion thereof) to a stored data point or dataset (e.g., a standard data point or data set, such as a stored blank spectra or background spectra); (2) a module to determine whether or not a change (e.g., a significant change) exists between the measured data and the stored data. In specific embodiments, such a microfludic device further comprises a module configured to put the microfluidic device back into an inactive or sleep mode (e.g., for a preset time period, or an adjusted time period), into active mode (e.g., detecting an analyte and/or undergoing a feedback application as described herein). In some embodiments, a return to an inactive mode or a sleep mode may occur if a change in data is detected, but is not determined to be significant enough to warrant a return to active mode.

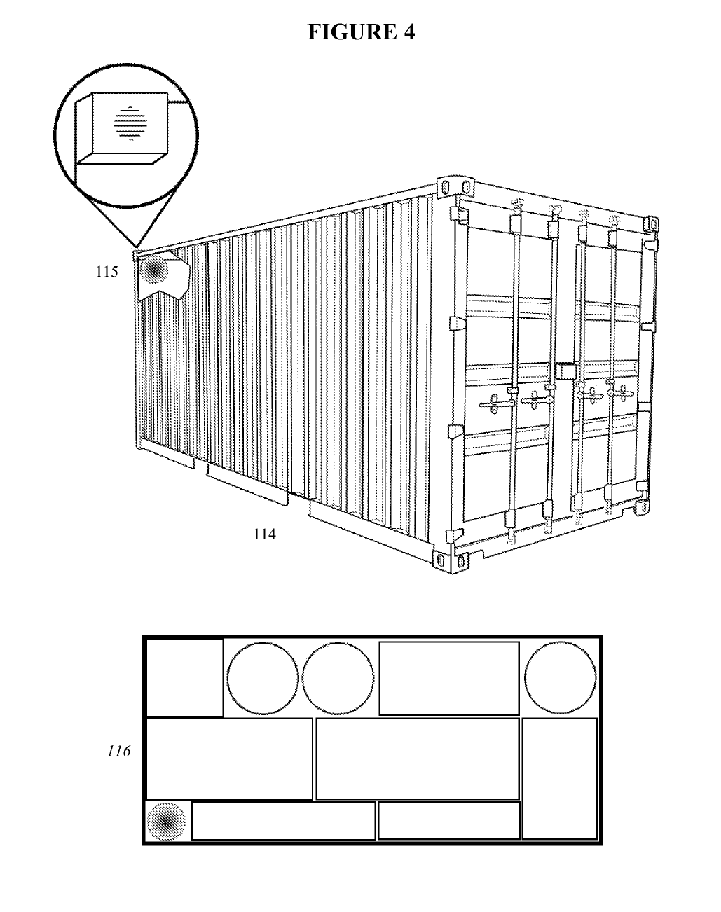

FIG. 4 illustrates deployment of the detection device in a space where long-term monitoring may necessitate intelligent duty cycling controls to save power and materials. In the instance of closed environments such as a cargo container (114), the device is located (115, 116) such that it is in constant contact with the fluid to be monitored (e.g., air) and responds to the presence of targeted analyte(s).

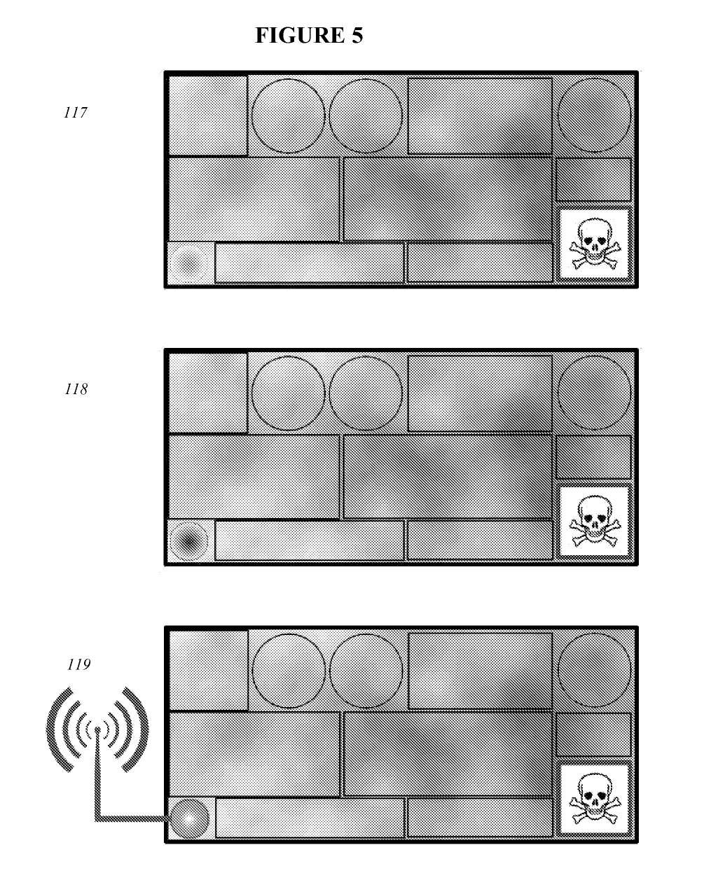

FIG. 5 illustrates an active evolution of the device shown in FIG. 4. Entry to the monitored space, presence of a targeted analyte (117), and/or periodic duty cycling enables (i.e., “wakes up”) the detection device for sensing (118). If the targeted analyte(s) are detected, a response is triggered, such as activation of an alarm or telemetering of data (119).

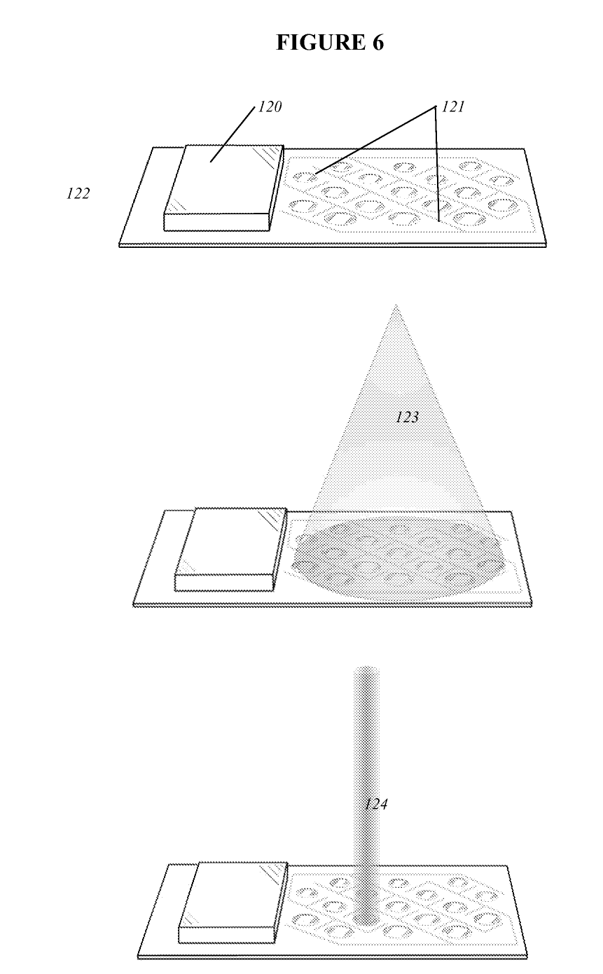

FIG. 6 illustrates a SERS sensor module having an integrated PROM containing a data library (e.g., of stored operating parameters, static parameters, analytical data, or combinations thereof) (120) and an array of active sites (121) in one embodiment (122). Intelligent controls enable interrogation of the active sites both collectively (123) or selectively (124), at one or more laser wavelengths or power levels, to obtain both singular and multiplexed data.

1. A microfluidic system for capturing and analyzing gas phase and/or airborne analytes in a liquid, comprising:
at least one liquid/gas interface site comprising at least a partially open microchamber, including at least one of a microchannel or a microcell,
at least one analytical instrument
at least one chemometric data processing module configured to process outputs from the analytical instrument; and,
at least one system control module configured to adjust one or more operating parameter;
wherein at least one operating parameter of the microfluidic system is adjusted based on an output from the chemometric processing module.

2. The microfluidic system of claim 1, further comprising at least one nanostructured surfaces in the microchamber or provision for the system to introduce nanostructured particles into the microchamber.

3. The microfluidic system of claim 1 wherein the analytical instrument is a spectrometer and the chemometric data processing module is configured to determine the chemical composition of analytes from spectrometer data.

4. The microfluidic system of claim 1 wherein the spectrometer is configured for SERS interrogation of analytes aggregated with nanostructures in the microchamber.

5. The microfluidic system of claim 1 wherein the system control module comprises an application to control the amount of selected analytes in the microchamber identified by the chemometric processor module by adjusting operating parameters of the system.

6. The microfluidic system of claim 1 wherein the parameter adjusting is accomplished by a feedback based on analyte data.

7. The microfluidic system of claim 1 wherein the operating parameters comprise at least one of
a. rate of condensation-evaporation cycling within microfluidic channel(s) and/or cell(s);
b. flowrate of colloid within microfluidic channels;
c. power level of interrogating laser;
d. wavelength of interrogating laser;
e. flow rate of sampled air (fluid in a gaseous phase);
f. integration time of the Raman spectrometer;
g. relative humidity of the sample gas;
h. nanoparticle size within colloid;
i. nanoparticle size deposited on the microchannel surface or substrate;
j. nanoparticle density deposited on the microchannel surface or substrate;
k. nanoparticle concentration in the working fluid;
l. chemical composition of working fluid, which may contain nanoparticles;
m. operating temperature;
n. background fluorescence spectra; and/or
o. photodecomposition fluorescence spectra.

8. The microfluidic system of claim 1 wherein the analytical instrument(s) comprise a surface enhanced vibrational spectrometer, a surface plasmon resonance spectrometer, a X-Ray spectrometer, an IR spectrometer, a visible light spectrometer, a UV spectrometer, an electromagnetic radiation absorption spectrometer, a mass spectrometer, a thermometer, a Raman spectrometer, or a combination thereof.

9. A process of detecting or measuring the amount of a gas phase and/or airborne analyte molecule in an air sample utilizing a microfluidic system, the process comprising:
providing at least one partially open microchamber comprising a liquid and a liquid surface that is in contact with the air sample;
exposing the sample of air with the surface of the liquid, thereby allowing analyte molecules to diffuse into the liquid in one or more of the microchambers;
interrogating the microcell with an analytical instrument to acquire data pertaining to the analyte molecules;
analyzing the acquired analytical instrument data with a chemometric data processing module; and,
adjusting at least one operating parameter of the microfluidic system based on an output from the chemometric data processing module.

10. The process of claim 9, wherein either the chamber and/or the liquid includes nanostructured material and whereinupon diffusion into the liquid, the analyte aggregates with and/or is deposited on the nanostructured material.

11. The process of claim 9 wherein the analytical instrument is a spectrometer and the chemometric analyzing step comprises determining chemical composition of analytes from spectrometer data.

12. The process of claim 9 wherein the interrogating step comprises performing SERS on analytes aggregated with nanostructures in the microchamber.

13. The process of claim 9 further comprising controlling the amount of selected analytes in the microchamber identified by the chemometric processor module as a result of adjusting the at least one operating parameter.

14. The process of claim 9 wherein the controlling analyte amount by adjusting parameters is a feedback process.

15. The process of claim 9 wherein the operating parameters comprise at least one of
a. rate of condensation-evaporation cycling within microfluidic channel(s) and/or cell(s);
b. flowrate of colloid within microfluidic channels;
c. power level of interrogating laser;
d. wavelength of interrogating laser;
e. flow rate of sampled air (fluid in a gaseous phase);
f. integration time of the Raman spectrometer;
g. relative humidity of the sample gas;
h. nanoparticle size within colloid;
i. nanoparticle size deposited on the microchannel surface or substrate;
j. nanoparticle density deposited on the microchannel surface or substrate;
k. nanoparticle concentration in the working fluid;
l. chemical composition of working fluid, which may contain nanoparticles;
m. operating temperature;
n. background fluorescence spectra; and/or
o. photodecomposition fluorescence spectra.

16. The process of claim 9 wherein the analytical instrument(s) comprise at least one of a surface enhanced vibrational spectrometer, a surface plasmon resonance spectrometer, a X-Ray spectrometer, an IR spectrometer, a visible light spectrometer, a UV spectrometer, an electromagnetic radiation absorption spectrometer, a mass spectrometer, a thermometer, a Raman spectrometer, or a combination thereof.
